(19) United States
(12) Patent Application Publication
(10) Pub. No.: US 2024/0408590 A1
(43) Pub. Date: Dec. 12, 2024

(54) BIODERIVED ORGANIC SOLVENTS

(71) Applicant: Fujifilm Electronic Materials U.S.A, Inc., N. Kingstown, RI (US)

(72) Inventors: Sanjay Malik, Attleboro, MA (US); William A. Reinerth, Riverside, RI (US); Binod B. De, Attleboro, MA (US); Stephanie Dilocker, Attleboro, MA (US); Raj Sakamuri, Sharon, MA (US)

(21) Appl. No.: 18/734,029

(22) Filed: Jun. 5, 2024

Related U.S. Application Data
(60) Provisional application No. 63/472,011, filed on Jun. 9, 2023.

(51) Int. Cl. B01J 41/02; B01J 41/10; B01J 47/02; G03F 7/004; G03F 7/32

(52) U.S. Cl. CPC B01J 41/02; B01J 41/10; B01J 47/02; G03F 7/0048; G03F 7/325

(57) ABSTRACT

The present disclosure is directed to methods and systems of purifying bioderived organic solvents. The purified bioderived organic solvents can be used in a multistep semiconductor manufacturing process.

### CROSS REFERENCE TO RELATED APPLICATIONS

The present application claims priority to U.S. Provisional Application Ser. No. 63/472,011, filed on Jun. 9, 2023, the contents of which are hereby incorporated by reference in their entirety.

### FIELD OF THE DISCLOSURE

The present disclosure relates to bioderived organic solvents, as well as systems and methods for purifying and/or using such solvents.

### BACKGROUND OF THE DISCLOSURE

Dielectric material requirements for semiconductor packaging applications are continuously evolving. The trend in electronic packaging continues to move towards faster processing speeds, increased complexity and higher packing density while maintaining a high level of reliability. As electronic packaging technology advances and chips continue to shrink in size, the demand for innovative, high performance, and environmentally friendly solvents is growing. Development and awareness of sustainable solvents are areas of growing interest in both the research and chemical industries where solvents can have a significant impact on pollution, energy usage, air quality, and climate changes. To make semiconductor manufacturing safer and more sustainable, it is desirable to utilize bioderived solvents that are safer, more efficient, and better for the environment than solvents that are currently used.

### SUMMARY OF THE DISCLOSURE

This disclosure is based on the unexpected discovery that certain purified bioderived organic solvents can be used in dielectric film-forming compositions to form dielectric films that possess a relatively low film shrinkage, relatively low dielectric constant and/or dissipation factor, and a relatively high glass transition temperature (Tg) (e.g., having a Tg higher than a solder paste reflow temperature (e.g., 260° C.)).

In one aspect, the present disclosure features methods of purifying a bioderived organic solvent, the method including: (1) passing the bioderived organic solvent through an ion exchange filter unit, wherein the ion exchange filter unit includes a housing and at least one first ion exchange filter and at least one second ion exchange filter in the housing, wherein the at least one first ion exchange filter and the at least one second ion exchange filter are both negatively charged ion exchange filters and are connected in series, and wherein the at least one first ion exchange filter is different from the at least one second ion exchange filter; (2) passing the bioderived organic solvent through at least one column containing an adsorbent to remove water in the bioderived organic solvent; and (3) distilling the bioderived organic solvent in a distillation column to obtain a purified bioderived organic solvent; wherein the bioderived organic solvent is obtained from a biological feedstock, the purified bioderived organic solvent includes an acid component (e.g., an organic acid and/or an inorganic acid) in an amount of from about 0.1 ppb by mass to about 1000 ppm by mass, and the purified bioderived organic solvent includes water in an amount of from about 0.1 ppb by mass to about 1000 ppm by mass.

In another aspect, the present disclosure features methods of purifying a bioderived organic solvent, the method including: (1) passing the bioderived organic solvent through at least one column containing an adsorbent to remove water in the bioderived organic solvent; (2) passing the bioderived organic solvent through an ion exchange filter unit, wherein the ion exchange filter unit includes a housing and at least one first ion exchange filter and at least one second ion exchange filter in the housing, wherein the at least one first ion exchange filter and at least one second ion exchange filter are both negatively charged ion exchange filters and are connected in series, and wherein the at least one first ion exchange filter is different from at least one second ion exchange filter; and (3) distilling the bioderived organic solvent in a distillation column to obtain a purified bioderived organic solvent; wherein the bioderived organic solvent is obtained from a biological feedstock, the purified bioderived organic solvent includes an acid component (e.g., an organic acid and/or an inorganic acid) in an amount of from about 0.1 ppb by mass to about 1000 ppm by mass, and the purified bioderived organic solvent include water in an amount of from about 0.1 ppb by mass to about 1000 ppm by mass.

In another aspect, the present disclosure features a purified bioderived organic solvent prepared from a purifying method described herein.

In another aspect, the present disclosure features a purified bioderived organic solvent, in which the bioderived organic solvent is obtained from a biological feedstock; the bioderived organic solvent includes an acid component in an amount of from about 0.1 ppb by mass to about 1000 ppm by mass; and the bioderived organic solvent includes water in an amount of from about 0.1 ppb by mass to about 1000 ppm by mass.

In another aspect, the present disclosure features methods of preparing a polymer, the methods including forming a polymer containing a polyimide precursor polymer, a polybenzoxazole precursor polymer, or a fully imidized polyimide polymer in a solvent system containing at least one first organic solvent and optionally, at least one second organic solvent, wherein the at least one first organic solvent includes a purified bioderived organic solvent described herein, the bioderived organic solvent is an aprotic polar solvent selected from the group consisting of lactones, ketones, ethers, alkyl aromatics, and alkyl alicyclics, and the at least one second organic solvent contains a carbonyl moiety.

In another aspect, the present disclosure features photosensitive compositions that include (1) at least one resin selected from the group consisting of epoxy resins, novolak resins, polyamide resins, polybenzoxazole precursor polymers, polyimide precursor polymers, and fully imidized polyimide polymers; and (2) a solvent system containing at least one first organic solvent and optionally, at least one second organic solvent; wherein the at least one first organic solvent includes a purified bioderived organic solvent described herein, the bioderived organic solvent is a solvent selected from the group consisting of lactones, ketones, ethers, alcohols, alkyl aromatics, and alkyl alicyclics, and the at least one second organic solvent includes a carbonyl group.

In another aspect, the present disclosure features dielectric film forming compositions including:
a) at least one dielectric polymer containing an epoxy resin, a novolak resin, a polyamide polymer, a polybenzoxazole precursor polymer, a polyimide precursor polymer, or a fully imidized polyimide polymer in a solvent system containing at least one first organic solvent and optionally at least one second organic solvent, wherein the at least one first organic solvent includes a purified bioderived organic solvent described herein, the bioderived organic solvent is a solvent selected from the group consisting of lactones, ketones, ethers, alcohols, alkyl aromatics, and alkyl alicyclics, and the at least one second organic solvent contains a carbonyl moiety;
b) at least one solubility switching compound (SSC);
c) at least one catalyst capable of inducing a polymerization reaction;
d) optionally at least one inorganic filler; and
e) optionally at least one metal-containing (meth)acrylate compound.

In another aspect, the present disclosure features dielectric film-forming compositions that include:
a) at least one cyanate ester compound, the at least one cyanate ester compound containing at least two cyanate groups;
b) at least one dielectric polymer including an epoxy resin, a novolak resin, a polyamide polymer, a polybenzoxazole precursor polymer, a polyimide precursor polymer, or a fully imidized polyimide polymer; and
c) at least one purified bioderived organic solvent described herein.

In another aspect, the present disclosure features dry films that include a carrier substrate and a dielectric film supported by the carrier substrate, in which the film is prepared from the dielectric film-forming composition described herein.

In another aspect, the present disclosure features pattern forming methods that include: (a) disposing a photosensitive composition described herein on a substrate to form a film; (b) exposing the film to radiation, heat, or a combination thereof; and (c) developing the exposed film using an organic developer to form a pattern on the substrate; wherein the organic developer includes a purified bioderived organic solvent described herein.

In another aspect, the present disclosure features processes that include:
a) depositing a dielectric film-forming composition described herein on a substrate to form a dielectric film;
b) exposing the dielectric film to radiation or heat or a combination of radiation and heat;
c) developing the exposed film by using an organic developer to form a patterned dielectric film having openings, wherein the organic developer includes a purified bioderived organic solvent described herein.

In another aspect, the present disclosure features processes for depositing a metal layer. These processes include:
a) depositing a dielectric film-forming composition described herein on a substrate to form a dielectric film;
b) exposing the dielectric film to radiation or heat or a combination of radiation and heat;
c) patterning the dielectric film to form a patterned dielectric film having openings;
d) optionally depositing a seed layer on the patterned dielectric film; and
e) depositing a metal layer in at least one opening in the patterned dielectric film.

In another aspect, the present disclosure features processes for forming a dielectric film on a substrate. These processes include:
a) providing a substrate containing conducting metal wire structures (which can contain copper) that form a network of lines and interconnects on the substrate;
b) depositing the dielectric film-forming composition described herein on the substrate to form a dielectric film; and
c) exposing the dielectric film to radiation or heat or a combination of radiation and heat.

In yet another aspect, the present disclosure features three-dimensional objects prepared by the process described herein. In some embodiments, the object includes the dielectric film in at least two or three stacks.

### DETAILED DESCRIPTION OF THE DISCLOSURE

In the present disclosure, “ppm” refers to “parts-per-million (10-6)”, “ppb” refers to “parts-per-billion (10-9)”, “ppt” refers to “parts-per-trillion (10-12)”. As defined herein, unless otherwise noted, all percentages expressed should be understood to be percentages by weight to the total weight of a composition. The term “solvent” mentioned herein, unless otherwise noted, refers to a single solvent or a combination of two or more (e.g., three or four) solvents.

### Bioderived Organic Solvents

In general, the present disclosure relates to purified bioderived organic solvents. As used herein, the “bioderived organic solvents” refers to organic solvents derived or obtained from a biological feedstock (e.g., biological, renewable materials). Examples of suitable biological feedstocks include lignocellulosic biomasses, sugar cane, corn, vegetable oils, oil wastes, or citrus wastes. In some embodiments, the bioderived organic solvents can include polar solvents (e.g., aprotic polar solvents or protic polar solvent).

Examples of suitable bioderived organic solvents include lactones (e.g., C₃-C₁₀ lactones), ketones (e.g., C₃-C₁₀ linear or cyclic ketones), ethers (e.g., C₃-C₁₀ ethers), alkyl aromatics (e.g., mono- and polycyclic aromatic hydrocarbons substituted by one or more C₁-C₁₀ alkyl groups), alcohols (e.g., C₁-C₁₀ alcohols), or alkyl alicyclics (e.g., mono- and polycyclic aliphatic hydrocarbons optionally containing one or more carbon-carbon double bonds and substituted by one or more C₁-C₁₀ alkyl groups). An example of a bioderived lactone is gamma-valerolactone, which can be derived from corn and sugar cane. An example of a bioderived ketone is cyrene, which can be derived from corn and sugar cane. An example of a bioderived ether is 2-methyl tetrahydrofuran, which can be derived from lignocellulosic biomasses. An example of a bioderived alcohol is glycerol, which can be derived from vegetable oils. Examples of bioderived alkyl alicyclics include limonenes (e.g., d-limonene) and pinenes (e.g., α-pinene), which can be derived from oil wastes or citrus wastes. Examples of bioderived alkyl alicyclics include cymenes (e.g., p-cymene), which can be derived from oil wastes or citrus wastes.

γ-Valerolactone (GVL) is a bioderived solvent. GVL is a 5 carbon (valero-) cyclic ester with 5 atoms (4 carbons and 1 oxygen) in the ring (γ-lactone). GVL is a colorless liquid stable at normal conditions and has a sweet, herbaceous odor, which makes it suitable to produce perfumes and food additives. There are multiple pathways to produce GVL. For example, biologically derived levulinic acid (LA) can be hydrogenated to produce γ-hydroxyvaleric acid, an unstable intermediate, which ring-closes by intramolecular esterification and loses a water molecule spontaneously to produce GVL. As another example, biologically derived LA can be dehydrated to form angelica lactone followed by hydrogenation to produce GVL. Thus, unpurified bioderived GVL may contain impurities such as formic acid, acetic acid, sulfuric acid, water, unconverted levulinic acid (LA), methyl tetrahydrofuran (MTHF), and 1,4 pentanediol, which may not be desirable for electronic applications.

In some embodiments, gamma-valerolactone can be prepared from biologically derived levulinic acid via catalytic hydrogenation using a non-acidic heterogeneous hydrogenation catalyst comprising a hydrogenation metal supported on a solid catalyst carrier. These and other methods of preparation of gamma-valerolactone have been described in, e.g., US Patent application 2010/0217038, U.S. Pat. Nos. 6,946,563; 6,617,464; 8,975,421; and 9,376,411, the contents of which are hereby incorporated by reference.

Dihydrolevoglucosenone (cyrene) is a polar solvent that can be derived in two simple steps from cellulose. See, e.g., Sherwood et al., Chemical Communications. 2014; 50 (68): 9650-9652. Cyrene demonstrates significant promise as a dipolar aprotic solvent for use in the methods described herein. The polarity of dihydrolevoglucosenone is similar to NMP, DMF and sulpholane.

### Water Content

In some embodiments, the bioderived organic solvents described herein can be substantially free of water or contain a relatively small amount of water. In some embodiments, the content of water in the bioderived organic solvents of the disclosure is from at least about 0.1 ppb (e.g., at least about 1 ppb, at least about 10 ppb, at least about 50 ppb, at least about 100 ppb, at least about 1 ppm, at least about 10 ppm, or at least about 100 ppm) by mass to at most about 1000 ppm (e.g., at most about 500 ppm, at most about 200 ppm, at most about 100 ppm, or at most about 50 ppm) by mass. In some embodiments, the amount of water in a solvent used for polymerization to form a polyimide polymer can be critical as the amount of water in the solvent during the polymerization process can affect the molecular weight of the resultant polymer. Without wishing to be bound by theory, it is believed that, when a bioderived organic solvent is used in formulations for coating, and the content of water is at least 10 ppm by mass, the adhesion of the film to a substrate such Si, SiOx, SiN, Cu and the like can be improved due to partial hydrolysis of siloxane compounds used as adhesion promoters. In addition, without wishing to be bound by theory, it is believed that, in a case where the content of water is 100 ppm by mass or less, the corrosion resistance of the bioderived organic solvent can be satisfactory. In some embodiments, the bioderived organic solvents described herein can be free of water. The content of water in the bioderived organic solvents can be measured by using a Karl Fischer moisture measurement method (coulometric titration) as a measurement principle.

### Trace Metal Components

In some embodiments, the bioderived organic solvents of the disclosure can be substantially free of metal components or contain a relatively small amount of certain metal components. The metal components can include at least one metal element selected from the group consisting of Na, K, Ca, Fe, Cu, Mg, Mn, Co, Al, Cr, Ni, Ti, Ag, and Zn. In some embodiments, the metal component can be in the form of an ion, a complex compound, a metal salt, an alloy, and the like. In some embodiments, the metal component can be in the form of a particle. In some embodiments, the metal component can be included in raw materials used during the production of the bioderived organic solvents or can be intentionally added to the bioderived organic solvents during or after their production.

In some embodiments, reducing the amount of trace metals in a bioderived organic solvent described herein can be important for optimizing performance when the solvent is used as a cast solvent where higher metal amounts can act as corrosives for devices. In some embodiments, the content of an individual metal component in the bioderived organic solvents of the disclosure is from at least about 10 ppt (e.g., at least about 100 ppt, at least about 500 ppt, at least about 1 ppb, or at least about 10 ppb) by mass to at most about 500 ppb (e.g., at most about 200 ppb, at most about 100 ppb, at most about 50 ppb, at most about 20 ppb, at most about 10 ppb, at most about 1 ppb, or at most about 100 ppt) by mass.

As used herein, when a bioderived organic solvent described herein contains two or more types of the metal components, the content of the metal components refers to a total content of the two or more types of metal components in the solvent. In some embodiments, the total content of all metal components in the bioderived organic solvents described herein is from at least about 50 ppt (e.g., at least about 100 ppt, at least about 1 ppb, at least about 5 ppb, or at least about 10 ppb) by mass to at most about 500 ppb (e.g., at most about 200 ppb, at most about 100 ppb, at most about 50 ppb, at most about 10 ppb, or at most about 1 ppb) by mass. The content of the metal components in the bioderived organic solvents described herein can be measured by using an inductively coupled plasma mass spectrometry (ICP-MS) method. Measurement of the content of the metal components by the ICP-MS method can be performed, for example, by using a device such as NexION350 available from PerkinElmer, Inc. (Walthan, MA).

Without wishing to be bound by theory, it is believed that, when the content of metal components is in the above range, generation of defects of a semiconductor device can be suppressed. In some embodiments, the bioderived organic solvents described herein can be free of metal components.

### Trace Acid Components

In some embodiments, the bioderived organic solvents described herein can be substantially free of acid components or contain a relatively small amount of acid components, such as organic acids (e.g., formic acid, acetic acid, or unconverted levulinic acid) or inorganic acids (e.g., sulfuric acid). In some embodiments, the acid components can be in an amount of at most about 1000 ppm (e.g., at most about 500 ppm, at most about 200 ppm, at most about 100 ppm, at most about 50 ppm, at most about 10 ppm, at most about 5 ppm, at most about 1 ppm, at most about 500 ppb, at most about 100 ppb, at most about 50 ppb, at most about 10 ppb, at most about 5 ppb, or at most about 1 ppb) by mass and at least about 0.1 ppb (e.g., at least about 0.5 ppb, at least about 1 ppb, at least about 5 ppb, or at least about 10 ppb) by mass of the bioderived organic solvents described herein.

### Purification Process of Bioderived Organic Solvent

In some embodiments, the present disclosure features methods of purifying a bioderived organic solvent. The method can include: (1) passing the bioderived organic solvent through an ion exchange filter unit; (2) passing the bioderived organic solvent through at least one column containing an adsorbent to remove water in the bioderived organic solvent; and (3) distilling the bioderived organic solvent in a distillation column to obtain a purified bioderived organic solvent.

In general, the above three steps can be performed in any sequence. For example, the above three steps can be performed in the sequence listed in the preceding paragraph. As another example, the purification process can be performed in the following sequence: passing the bioderived organic solvent through at least one column containing an adsorbent, distilling the bioderived organic solvent, and passing the bioderived organic solvent through an ion exchange filter. As a further example, the purification process can be performed in the following sequence: passing the bioderived organic solvent through at least one column containing an adsorbent, passing the bioderived organic solvent through an ion exchange filter, and distilling the bioderived organic solvent.

In some embodiments, the purification processes described herein can use at least one (e.g., two or three) column (e.g., a dehydration column) containing an adsorbent to remove moisture or certain other impurities in a bioderived organic solvent. In some embodiments, the adsorbent in such a column can include a molecular sieve (e.g., zeolite 3A, zeolite 4A, or zeolite 5A), a silica gel, activated alumina, activated carbon or an ion exchange resin. In some embodiments, when one column containing an adsorbent is not sufficient to lower moisture in the bioderived organic solvent to a desired level (e.g., at most about 1000 ppm), two or more of such columns can be used. In such embodiments, these columns can be in fluid communication with each other and are connected in series.

In some embodiments, the purification processes described herein can use at least one (e.g., two or three) ion exchange filter unit to remove metal impurities from a bioderived organic solvent. In some embodiments, the ion exchange filter unit can include a housing, and at least one (e.g., 2, 3, 4, or 5) first ion exchange filter and at least one (e.g., 2, 3, 4, or 5) second ion exchange filter in the housing. In some embodiments, the at least one first ion exchange filter and the at least one second ion exchange filter are both negatively charged ion exchange filters or cationic ion exchange filters (i.e., including one or more filtration medium containing a negatively charged ion exchange resin) and are connected in series. When there are more than one first ion exchange filter, the multiple first ion exchange filters can be connected in parallel to increase flow rate and productivity. When there are more than one second ion exchange filter, the multiple second ion exchange filters can be connected in parallel to increase flow rate and productivity.

In general, the at least one first ion exchange filter is different from the at least one second ion exchange filter (e.g., containing different filtration media). In some embodiments, the at least one first ion exchange filter can be capable of primarily removing heavy metals (e.g., Fe, Ni, Cr, Zn, or Cu), while the at least one second ion exchange filter can be capable of primarily removing alkali or alkaline earth metals (e.g., K, Na, or Ca). In some embodiments, the first ion exchange filter can be capable of removing at least about 90 wt % (e.g., at least about 92 wt % or at least about 95 wt %) of one or more heavy metals and/or at most about 10 wt % (e.g., at most about 8 wt % or at most about 5 wt %) of one or more alkali or alkaline earth metals in a bioderived organic solvent. In some embodiments, the second ion exchange filter can be capable of removing at least about 70 wt % (e.g., at least about 75 wt %, at least about 80 wt %, at least about 85 wt %, or at least about 90 wt %) one or more alkali or alkaline earth metals and/or at most about 30 wt % (e.g., at most about 25 wt %, at most about 20 wt %, at most about 15 wt %, or at most about 10 wt %) one or more heavy metals in a bioderived organic solvent.

In some embodiments, it is preferable that the at least one second ion exchange filter is disposed downstream of the at least one first ion exchange filter. Without wishing to be bound by theory, it is believed that, in such embodiments, heavy metals in the organic solvent can be removed first, which facilitates the removal of alkali or alkaline earth metals because residual heavy metals in the organic solvent may impede the removal of alkali or alkaline earth metals by the at least one second ion exchange filter. In some embodiments, the sequence of the at least one first and second ion exchange filters can be reversed.

In some embodiments, the at least one first or second ion exchange filters can include one or more negatively charged ion exchange resin membranes as a filtration medium to remove positively charged particles and/or cationic metal ions from the organic solvent. The negatively charged ion-exchange resin membrane used in the present disclosure is not particularly limited, and filters including an ion exchange resin having a suitable ion-exchange group immobilized to a resin membrane can be used. Examples of such ion-exchange resin membranes include strongly acidic cation-exchange resins having a cation-exchange group (such as a sulfonic acid or sulfonate group) chemically modified on the resin membrane. Examples of suitable resin membranes include those containing cellulose, diatomaceous earth, a polyamide (e.g., nylon), a polyolefin (such as polyethylene (e.g., high density polyethylene or ultra high molecular weight polyethylene), polypropylene, or polystyrene), a resin having an imide group, a resin having an amide group and an imide group, a fluoropolymer (e.g., a polytetrafluoroethylene or a perfluoroalkoxy alkane polymer), or a copolymer or combination thereof. In some embodiments, the ion-exchange resin membrane can be a membrane having an integral structure of a particle-removing membrane and an ion-exchange resin membrane. Polyalkylene (e.g., PE, PP, or PTFE) membranes with a cation-exchange group (e.g., a sulfonate group) chemically modified thereon are preferred. Filters with cation-exchange resin membranes used in the present disclosure can be commercially available filters with metal ion removal functionality. Commercial example of such a cation-exchange filter include lonKleen filters available from Pall Corporation (Port Washington, NY) and Protego Plus filters available from Entegris (Billerica, MA). These filters can be selected based on the ion exchange efficiency and have an estimated pore size in the range of about 100 nm to about 500 nm.

Examples of the shape of the membrane material in the at least one first or second ion exchange filters include a pleated type, a flat membrane type, a hollow fiber type, a porous body as described in JP-A No. 2003-112060 and the like. In some embodiments, when the ion exchange membrane has porosity, it is also possible to remove at least a portion of fine particles in the bioderived organic solvent.

In some embodiments, the at least one first ion exchange filter can include a filtration medium that includes a polyethylene having sulfonate groups, a polytetrafluoroethylene having sulfonate groups, or a copolymer thereof, which is capable of primarily removing heavy metals (e.g., Fe, Ni, Cr, Zn, or Cu). A commercial example of such a filter is an lonKleen filter (such as lonKleen SL filters) from Pall Corporation (Port Washington, NY). In some embodiments, the ion exchange filter unit described herein can include two or three such first ion exchange filters connected in parallel to increase productivity.

In some embodiments, the at least one second ion exchange filter can include a filtration medium that includes a polyethylene having sulfonate groups, a polytetrafluoroethylene having sulfonate groups, or a copolymer thereof, which is capable of primarily removing alkali or alkaline earth metals (e.g., K, Na, or Ca). Commercial examples of such a filter include Protego Plus filters (such as Protego Plus IPA filters) and Protego AT 5 nm/lonex combo filters available from Entegris (Billerica, MA). In some embodiments, the ion exchange filter unit described herein can include two or three such second ion exchange filters connected in parallel to increase productivity.

Without wishing to be bound by theory, it is believed that including two different types of ion exchange filters in the ion exchange filter unit described herein can significantly reduce the amount of metal impurities (e.g., to a total amount of at most about 200 ppt) in the purified organic solvent, compared to a system in which only one type of ion exchange filter is used.

In some embodiments, the purification process described herein can use at least one (e.g., two or three) distillation column to purify a bioderived organic solvent. In general, such a distillation column can be any suitable distillation column known in the art and can be used to purify the bioderived organic solvent through distillation to remove the majority of the organic and metal impurities and particles.

In some embodiments, the purification process described herein can use at least one (e.g., two or three) additional filter unit (i.e., located and upstream or downstream of the ion exchange filter unit described above) to purify a bioderived organic solvent. In some embodiments, each additional filter unit can include a filter housing and one or more filters in the filter housing. The additional filter units can be different in functionality or property and offer different purification treatments. In some embodiments, each additional filter unit can independently be selected from the group consisting of a particle removal filter, an ion exchange filter, and an ion absorption filter.

In some embodiments, the pre-processed or unpurified bioderived organic solvent can have a purity of at most about 99% (e.g., at most about 98%, at most about 97%, at most about 96%, or at most about 95%). In some embodiments, the post-processed or purified bioderived organic solvent obtained from the methods described herein can have a purity of at least about 99.5% (e.g., at least about 99.9%, at least about 99.95%, at least about 99.99%, at least about 99.995%, at least about 99.999%, at least about 99.9995%, at least about 99.9999%, or 100%). As mentioned herein, “purity” refers to the weight percentage of the solvent in the total weight of the liquid. The content of the organic solvent in a liquid can be measured by using a gas chromatography mass spectrometry (GC-MS) device (e.g., a thermal desorption (TD) GC-MS device).

Other methods of purification of organic solvents have been described in, e.g., U.S. Patent applications US202103008; US2021060526; US2021220754; and US2021300851, the contents of which are hereby incorporated by reference.

### Polymerization Process

In some embodiments, the present disclosure features methods (e.g., a polymerization method) of forming a polymer using the bioderived organic solvents described herein. In some embodiments, the method can include forming a polymer containing a polyimide (PI) precursor polymer, a polybenzoxazole (PBO) precursor polymer, or a fully imidized polyimide in a solvent system containing at least one first organic solvent and optionally, at least one second organic solvent. In some embodiments, the at least one first organic solvent can include one or more the bioderived organic solvents described herein (e.g., an aprotic polar solvent). In some embodiments, the at least one second organic solvent can include a solvent (e.g., a non-aqueous solvent) containing a carbonyl group.

Examples of the second organic solvent suitable for the polymerization method described herein include, but are not limited to, 1-butylpyrrolidin-2-one (e.g., Tamisolve NxG), N-methyl-2-pyrrolidone (NMP), N-ethyl-2-pyrrolidone (NEP), 1,3-dimethyl-2-imidazolidinone (DMI), γ-butyrolactone (GBL), δ-valerolactone (δ-VL), ε-caprolactone (ε-CL), 3-dimethyl-2-imidazolidinone, 3-methoxy-N,N-dimethylpropanamide, 3-methoxy-N,N-dibutylpropanamide, cyclohexanone (CH), cyclopentanone (CP), isophorone (IP), propylene carbonate (PC), ethylene carbonate (EC), and the like.

In some embodiments, in the preparation of polyimide precursor polymers, polybenzoxazole precursor polymers, or fully imidized polyimide polymers, mixed solvent systems containing at least one bioderived organic solvent and at least one second organic solvent can be used. In some embodiments, such a solvent system can prevent a change of liquid characteristics such as viscosity, due to absorption of moisture by the polymer formed in the reaction. The polyimide precursor, polybenzoxazole precursor, or fully imidized polyimide polymer thus formed can be excellent in film preparation properties (coating properties) and preservation stability. In some embodiments, even when such a composition (e.g., a coating liquid) is stirred while being exposed under an air atmosphere, as in a roll type coating device and the like, an increase in viscosity of the composition can be avoided and uniform film forming can be achieved.

### Photosensitive Compositions

In some embodiments, the present disclosure features photosensitive compositions containing at least one resin and at least one first organic solvent (e.g., at least one bioderived organic solvent) described herein and optionally at least one second organic solvent described herein. As used herein, the terms “resin” and “polymer” are used interchangeably.

In some embodiments, the resins suitable for the photosensitive compositions described herein can include at least one (e.g., two, three, or four) dielectric polymer containing an epoxy resin, a novolak resin, a polyamide resin, a polybenzoxazole precursor polymer, a polyimide precursor polymer, or fully imidized polyimide polymer. The photosensitive compositions described herein can be compositions that are radiation sensitive in actinic ray or similar ray. In some embodiments, the dielectric polymer is a fully imidized polyimide polymer. The fully imidized polyimide polymer mentioned herein is at least about 90% (e.g., at least about 95%, at least about 98%, at least about 99%, or about 100%) imidized.

In some embodiments, the weight average molecular weight of the fully imidized polymer is at least about 20,000 Daltons (e.g., at least about 25,000 Daltons, at least about 30,000 Daltons, at least about 35,000 Daltons, at least about 40,000 Daltons, at least about 45,000 Daltons, at least about 50,000 Daltons, or at least about 55,000 Daltons) and/or at most about 100,000 Daltons (e.g., at most about 95,000 Daltons, at most about 90,000 Daltons, at most about 85,000 Daltons, at most about 80,000 Daltons, at most about 75,000 Daltons, at most about 70,000 Daltons, at most about 65,000 Daltons, or at most about 60,000 Daltons).

In some embodiments, the at least one (e.g., two, three, or four) fully imidized polyimide polymer is prepared by reaction of at least one diamine with at least one tetracarboxylic acid dianhydride. In some embodiments, the resulting polymer is soluble in the bioderived organic solvent of this disclosure to facilitate the formation of a dielectric film with a planarized surface (e.g., the difference in the highest and lowest points on a top surface of the dielectric film is less than about 2 microns). Examples of fully imidized polyimide polymers are known in the art and have been described, e.g., in U.S. Application Publication No. 2019/0077913, the entire contents of which are hereby incorporated by reference.

Methods to synthesize end-capped and non-endcapped PI precursor polymers are well known to those skilled in the art. Examples of such methods and PI precursor polymers are disclosed in, e.g., U.S. Pat. Nos. 2,731,447, 3,435,002, 3,856,752, 3,983,092, 4,026,876, 4,040,831, 4,579,809, 4,629,777, 4,656,116, 4,960,860, 4,985,529, 5,006,611, 5,122,436, 5,252,534, 5,478,915, 5,773,559, 5,783,656, 5,969,055, 9,617,386, and US application publication numbers US2004/0265731, US2004/0235992, and US2007/0083016, the entire contents of which are hereby incorporated by reference.

Methods to synthesize polybenzoxazole precursor polymers are known to those skilled in the art. Examples of such methods and PBO precursor polymers are disclosed in, e.g., U.S. Pat. Nos. 6,143,467, 7,195,849, 7,129,011, and 9,519,216, the entire contents of which are hereby incorporated by reference.

Methods to synthesize polyimide precursor polymer (e.g. polyamic acid ester polymers) are also known to those skilled in the art. Examples of such methods and PI precursor polymers are disclosed in, e.g., U.S. Pat. Nos. 4,040,831, 4,548,891, 5,834,581 and 6,511,789, the entire contents of which are hereby incorporated by reference.

Examples of suitable epoxy resin used as dielectric film materials are known to those skilled in the art. Example of such resins are disclosed in, e.g., U.S. Pat. No. 4,882,245 and U.S. Patent Application No. 2006/0257785, the entire contents of which are hereby incorporated by reference.

Examples of suitable Novolak resin used as dielectric film materials are known to those skilled in the art. Example of such resins are disclosed in U.S. Pat. Nos. 5,413,894; 5,306,594 and 4,959,292, the entire contents of which are hereby incorporated by reference.

In some embodiments, the photosensitive compositions described herein can be dielectric film-forming compositions and the resin described herein can be dielectric polymers. In some embodiments, the dielectric film-forming composition described herein can include at least one (e.g., two, three, or four) cyanate ester compound. In some embodiments, the cyanate ester compound can include at least two cyanate groups. Without wishing to be bound by theory, it is believed that the cyanate ester compound can be cyclized and/or crosslinked thermally (e.g., with or without a catalyst) to form an interpenetrating network with the dielectric polymer. Further, without wishing to be bound by theory, it is believed that including a cyanate ester compound in the dielectric film-forming composition described herein can lower the dielectric constant (K) and/or dissipation factor (DF) of the film formed from the composition. Examples of suitable cyanate ester compounds have been described, e.g., in U.S. Application Publication No. 2022/0127459, the entire contents of which are hereby incorporated by reference.

In some embodiments, the amount of the at least one cyanate ester compound is at least about 0.1 weight % (e.g., at least about 0.5 weight %, at least about 1.0 weight %, at least about 2.0 weight %, or at least 2.5 weight %) and/or at most about 10 weight % (e.g., at most about 5 weight %, at most about 7 weight %, or at most about 9 weight) of the total weight of the photosensitive composition (e.g., a dielectric film-forming composition) described herein.

In some embodiments, the dielectric film-forming composition described herein can include at least one (e.g., two, three, or four) dielectric polymer selected from the group consisting of polybenzoxazole precursor polymers, polyimide precursor polymers, and fully imidized polyimide polymers, such as those described herein. In some embodiments, the dielectric polymer is a fully imidized polyimide polymer. The preferred fully imidized polyimide polymers are those without having any polymerizing moiety attached to the polymer. Without wishing to be bound by theory, it is believed that including the above polymers in the dielectric film-forming composition described herein can increase the glass transition temperature, decrease the thermal shrinkage, and improve the mechanical properties of the film formed by the composition.

In some embodiments, the amount of the resin or dielectric polymer is at least about 2 weight % (e.g., at least about 5 weight %, at least about 10 weight %, at least about 15 weight %, or at least about 20 weight %) and/or at most or about 55 weight % (e.g., at most about 50 weight %, at most about 45 weight %, at most about 40 weight %, at most about 35 weight %, at most about 30 weight %, or at most about 25 weight %) of the total weight of the photosensitive composition (e.g., a dielectric film-forming composition) described herein.

In some embodiments, the dielectric film-forming compositions described herein can further include a solvent system containing at least one first organic solvent and optionally, at least one second organic solvent. In some embodiments, the first organic solvent can be a purified bioderived organic solvent described herein (e.g., an aprotic polar solvent selected from the group consisting of lactone, ketone, ether, alkyl aromatic, and alkyl alicyclic solvents). In some embodiments, the second organic solvent is a solvent preferably containing a carbonyl group. In some embodiments, the bioderived organic solvent used in the dielectric film-forming composition is gamma-valerolactone, cyrene, or 2-methyl tetrahydrofuran.

Examples of the second organic solvents suitable for the dielectric film-forming compositions described herein include, but are not limited to, alkylene carbonates such as ethylene carbonate, propylene carbonate, butylene carbonate, and glycerine carbonate; lactones such as gamma-butyrolactone, ε-caprolactone, γ-caprolactone and γ-valerolactone; cycloketones such as cyclopentanone and cyclohexanone; linear ketones such as methyl ethyl ketone (MEK) and methyl isobutyl ketone (MIBK); esters such as n-butyl acetate; ester alcohol such as ethyl lactate; ether alcohols such as tetrahydrofurfuryl alcohol; glycol esters such as propylene glycol methyl ether acetate; glycol ethers such as propylene glycol methyl ether (PGME); cyclic ethers such as tetrahydrofuran (THF); and pyrrolidones such as N-methyl-2-pyrrolidone, N-ethyl-2-pyrrolidone or N-butyl-2-pyrrolidone or TamiSolve™ NxG; and dialkyl sulfoxide such as dimethyl sulfoxide.

In some embodiments, the total amount of the solvent (e.g., the first and second organic solvents) is at least about 20 weight % (e.g., at least about 25 weight %, at least about 30 weight %, at least about 35 weight %, at least about 40 weight %, at least about 45 weight %, at least about 50 weight %, at least about 55 weight %, at least about 60 weight %, or at least about 65 weight %) and/or at most about 98 weight % (e.g., at most about 95 weight %, at most about 90 weight %, at most about 85 weight %, at most about 80 weight %, at most about 75 weight %, at most about 70 weight %, or at most about 60 weight %) of the total weight of the photosensitive composition (e.g., a dielectric film-forming composition) described herein.

In some embodiments, the dielectric film-forming composition of this disclosure can optionally include at least one (e.g., two, three, or four) catalyst (e.g., an initiator). In some embodiments, depending on the type of the catalyst used, the catalyst is capable of cyclizing and/or crosslinking of cyanate ester, or inducing crosslinking or polymerization reactions when exposed to heat (e.g., a thermal initiator) and/or a source of radiation (e.g., a photoinitiator such as free radical initiator).

In some embodiments, the dielectric film-forming composition described herein can optionally include at least one (e.g., two, three, or four) cyanate curing catalyst to facilitate the curing of the cyanate ester compound (e.g., to form an interpenetrating network) and/or reduce curing temperature of dielectric film. The cyanate curing catalyst can be in either a photosensitive dielectric film-forming composition or a non-photosensitive dielectric film-forming composition.

In some embodiments, the cyanate curing catalyst can be selected from the group consisting of metal carboxylate salts and metal acetylacetonate salts. The metal in the metal carboxylate salts and metal acetylacetonate salts can be selected from the group consisting of zinc, copper, manganese, cobalt, iron, nickel, aluminum, titanium, zirconium, and mixtures thereof. Examples of cyanate curing catalysts include metal salts such as zirconyl dimethacrylate, zinc octanoate, zinc naphthenate, cobalt naphthenate, copper naphthenate, and acetylacetone iron; phenol compounds such as octylphenol and nonylphenol; alcohols such as 1-butanol and 2-ethylhexanol; imidazole compounds such as 2-methylimidazole, 2-ethyl-4-methylimidazole, 2-phenylimidazole, 1-cyanoethyl-2-phenylimidazole, 1-cyanoethyl-2-ethyl-4-methylimidazole, 2-phenyl-4,5-dihydroxymethylimidazole, and 2-phenyl-4-methyl-5-hydroxymethylimidazole; amine compounds such as dicyandiamide, benzyldimethylamine, and 4-methyl-N,N-dimethylbenzylamine; phosphorus compounds such as phosphine compounds and phosphonium compounds; epoxy-imidazole adduct compounds; and peroxides such as benzoyl peroxide, p-chlorobenzoyl peroxide, di-t-butyl peroxide, diisopropyl peroxycarbonate, and di-2-ethylhexyl peroxycarbonate. These catalysts are commercially available. Examples of the commercially available catalysts include Amicure PN-23 (trade name, manufactured by Ajinomoto Fine-Techno Co., Inc.), Novacure HX-3721 (trade name, manufactured by Asahi Kasei Corporation), and Fujicure FX-1000 (trade name, manufactured by Fuji Kasei Kogyo Co., Ltd.). One or a combination of two or more of these catalysts can be used in the composition described herein. Other examples of such catalysts have been described in, e.g., U.S. Patent Application number 2018/0105488 and U.S. Pat. No. 9,822,226, the contents of which are hereby incorporated by reference.

In some embodiments (e.g., in a photosensitive composition), the dielectric film-forming composition described herein can optionally include at least one (e.g., two, three, or four) photoinitiator to facilitate crosslinking reactions of a crosslinker (e.g., a reactive functional compound described herein) or crosslinking reactions between a crosslinker and the dielectric polymer (e.g., when it includes a cross-linkable group). Specific examples of photoinitiators include, but are not limited to, 1,8-octanedione, 1,8-bis[9-(2-ethylhexyl)-6-nitro-9H-carbazol-3-yl]-1,8-bis(O-acetyloxime), 2-hydroxy-2-methyl-1-phenylpropan-1-one, 1-hydroxycyclohexyl phenyl ketone (Irgacure 184 from BASF), a blend of 1-hydroxycyclohexylphenylketone and benzophenone (Irgacure 500 from BASF), 2,4,4-trimethylpentyl phosphine oxide (Irgacure 1800, 1850, and 1700 from BASF), 2,2-dimethoxyl-2-acetophenone (Irgacure 651 from BASF), bis(2,4,6-trimethyl benzoyl)phenyl phosphine oxide (Irgacure 819 from BASF), 2-methyl-1-[4-(methylthio)phenyl]-2-morphorinopropane-1-one (Irgacure 907 from BASF), (2,4,6-trimethylbenzoyl)diphenyl phosphine oxide (Lucerin TPO from BASF), 2-(Benzoyloxyimino)-1-[4-(phenylthio)phenyl]-1-octanone (Irgacure OXE-01 from BASF), 1-[9-ethyl-6-(2-methylbenzoyl)-9H-carbazol-3-yl]ethanone, 1-(O-acetyloxime) (Irgacure OXE-2 from BASF), ethoxy (2,4,6-trimethylbenzoyl)phenyl phosphine oxide (Lucerin TPO-L from BASF), a blend of phosphine oxide, hydroxy ketone and a benzophenone derivative (ESACURE KTO46 from Arkema), 2-hydroxy-2-methyl-1-phenylpropane-1-one (Darocur 1173 from Merck), NCI-831 (ADEKA Corp.), NCI-930 (ADEKA Corp.), N-1919 (ADEKA Corp.), benzophenone, 2-chlorothioxanthone, 2-methylthioxanthone, 2-isopropylthioxanthone, benzodimethyl ketal, 1,1,1-trichloroacetophenone, diethoxyacetophenone, m-chloroacetophenone, propiophenone, anthraquinone, dibenzosuberone and the like.

In some embodiments, a photosensitizer can be used in the dielectric film-forming composition where the photosensitizer can absorb light in the wavelength range of 193 to 405 nm. Examples of photosensitizers include, but are not limited to, 9-methylanthracene, anthracenemethanol, acenaphthylene, thioxanthone, methyl-2-naphthyl ketone, 4-acetylbiphenyl, and 1,2-benzofluorene.

Specific examples of thermal initiators include, but are not limited to, benzoyl peroxide, cyclohexanone peroxide, lauroyl peroxide, tert-amyl peroxybenzoate, tert-butyl hydroperoxide, di(tert-butyl) peroxide, dicumyl peroxide, cumene hydroperoxide, succinic acid peroxide, di(n-propyl) peroxydicarbonate, 2,2-azobis(isobutyronitrile), 2,2-azobis(2,4-dimethylvaleronitrile), dimethyl-2,2-azobisisobutyrate, 4,4-azobis(4-cyanopentanoic acid), azobiscyclohexanecarbonitrile, 2,2-azobis(2-methylbutyronitrile) and the like.

In some embodiments, the amount of the catalyst is at least about 0.2 weight % (e.g., at least about 0.5 weight %, at least about 0.8 weight %, at least about 1.0 weight %, or at least about 1.5 weight %) and/or at most about 3.0 weight % (e.g., at most about 2.8 weight %, at most about 2.6 weight %, at most about 2.3 weight %, or at most about 2.0 weight %) of the total weight of the photosensitive composition (e.g., a dielectric film-forming composition) described herein.

In some embodiments, the dielectric film-forming composition described herein can optionally include at least one (e.g., two, three, or four) reactive functional compound. In some embodiments, the reactive functional compound can include at least two functional groups (e.g., (meth)acrylate, alkenyl, or alkynyl groups). In some embodiments, the functional groups on the reactive functional compound are capable of reacting with another molecule of the reactive functional compound or with the dielectric polymer (e.g., when it includes a cross-linkable group). Without wishing to be bound by theory, it is believed that the reactive functional compound can be used as a crosslinker in a photosensitive composition to form a negative photosensitive film.

In some embodiments, the reactive functional compound is a compound containing at least two (meth)acrylate groups. As used herein, the term “(meth)acrylate” include both acrylates and methacrylates. Examples of such compounds include, but are not limited to, 1,3-butylene glycol di(meth)acrylate, 1,4-butanediol di(meth)acrylate, 1,5-pentanediol di(meth)acrylate, 1,6-hexanediol di(meth)acrylate, 1,9-nonanediol di(meth)acrylate, 1,10-decanediol di(meth)acrylate, 1,12-dodecanediol di(meth)acrylate, tetraethyleneglycol di(meth)acrylate, cyclohexane dimethanol di(meth)acrylate, polyethylene glycol di(meth)acrylate, ethylene glycol di(meth)acrylate, polyethylene glycol di(meth)acrylate, diurethane di(meth)acrylate, 1,4-phenylene di(meth)acrylate, 2,2-bis[4-(2-hydroxy-3-methacryloxypropoxy)phenyl]propane, bis(2-hydroxyethyl)-isocyanurate di(meth)acrylate, neopentyl glycol di(meth)acrylate, tricyclodecane dimethanol di(meth)acrylate, propoxylated (3) glycerol tri(meth)acrylate, pentaerythritol tetra(meth)acrylate, pentaerythritol tri(meth)acrylate, dipentaerythritol penta-/hexa-(meth)acrylate, isocyanurate tri(meth)acrylate, ethoxylated glycerine tri(meth)acrylate, trimethylol propane tri(meth)acrylate, ditrimethylol propane tetra(meth)acrylate, ethoxylated pentaerythritol tetra(meth)acrylate, tetramethylol methane tetra(meth)acrylate, 1,2,4-butanetriol tri(meth)acrylate, diglycerol tri(meth)acrylate, trimethylol propane ethoxylate tri(meth)acrylate, trimethylol propane polyethoxylate tri(meth)acrylate, tetramethylolmethane tetra(meth)acrylate and tris(2-hydroxyethyl) isocyanurate triacrylate. The preferred reactive functional compounds are di(meth)acrylate of an unsubstituted/substituted linear, branch or cyclic C₁-C₁₀ alkyl or an unsubstituted/substituted aromatic group. The reactive functional compound can be used alone or in combination of two or more kinds thereof in the dielectric film-forming composition described herein.

In some embodiments, the amount of the at least one reactive functional compound is at least about 1 weight % (e.g., at least about 2 weight %, at least about 3 weight %, at least about 4 weight %, or at least 5 weight %) and/or at most about 25 weight % (e.g., at most about 20 weight %, at most about 15 weight %, at most about 10 weight %, or at most about 8 weight %)) of the total weight of the photosensitive composition (e.g., a dielectric film-forming composition) described herein.

In some embodiments, the dielectric film-forming composition can optionally contain at least one mono (meth)acrylate containing compound. In some embodiment, the at least one mono (meth)acrylate containing compound is selected from the group consisting of bornyl acrylate, isobornyl acrylate, dicyclopentenyloxyethyl acrylate, dicyclopentenylacrylate, dicyclopentenyloxyethyl methacrylate, dicyclopentenyl methacrylate, bicyclo[2.2.2]oct-5-en-2-yl acrylate, 2-[(bicyclo[2.2.2]oct-5-en-2-yl)oxy]ethyl acrylate, 3a,4,5,6,7,7a-hexahydro-1H-4,7-ethanoinden-6-yl acrylate, 2-[(3a,4,5,6,7,7a-hexahydro-1H-4,7-ethanoinden-6-yl)oxy]ethyl acrylate, tricyclo[5,2,1,0^{2,6}]decyl acrylate, and tetracyclo[4,4,0,1^{2,5}, 1^{7,10}]dodecanyl acrylate. Without wishing to be bound by theory, it is believed that including at least one mono (meth)acrylate containing compound can enhance the mechanical properties of the film formed by the dielectric film-forming composition described herein (e.g., by forming a polymer and/or reacting (or crosslinking) with the reactive functional compound).

In some embodiments, the dielectric film-forming composition optionally includes other optional components, such as one or more (e.g., two, three, or four) inorganic fillers, adhesion promoters, surfactants, copper passivating agents, plasticizers, antioxidants, dyes, and/or colorants. Examples of such components have been described, e.g., in U.S. Application Publication No. 2022/0127459, the entire contents of which are hereby incorporated by reference.

In some embodiments, a dielectric film can be prepared from a dielectric film-forming composition of this disclosure by a process containing the steps of: (a) coating the dielectric film-forming composition described herein on a substrate (e.g. a semiconductor substrate) to form a dielectric film; and (b) optionally baking the film at an elevated temperature (e.g., from about 50° C. to about 150° C.) for a period of time (e.g., from about 20 seconds to about 600 seconds).

Coating methods for preparation of the dielectric film include, but are not limited to, (1) spin coating, (2) spray coating, (3) roll coating, (4) rod coating, (5) rotation coating, (6) slit coating, (7) compression coating, (8) curtain coating, (9) die coating, (10) wire bar coating, (11) knife coating and (12) lamination of dry film. In case of coating methods (1)-(11), the dielectric film-forming composition is typically provided in the form of a solution. One skilled in the art would choose the appropriate solvent type and solvent concentration based on the coating type.

Substrates can have circular, square or rectangular shapes such as wafers or panels in various dimensions. Examples of suitable substrates are epoxy molded compound (EMC), silicon, glass, copper, stainless steel, copper cladded laminate (CCL), aluminum, silicon oxide and silicon nitride. Substrates can be flexible such as polyimide, PEEK, polycarbonate, and polyester films. Substrates can have surface mounted or embedded chips, dyes, or packages. Substrates can be sputtered or pre-coated with a combination of seed layer and passivation layer. In some embodiments, the substrates mentioned herein can be a semiconductor substrate. As used herein, a semiconductor substrate is a substrate (e.g., a silicon or copper substrate or wafer) that becomes a part of a final electronic device.

The thickness of the dielectric film of this disclosure is not particularly limited. In some embodiments, the dielectric film has a film thickness of at least about 1 micron (e.g., at least about 2 microns, at least about 3 microns, at least about 4 microns, at least about 5 microns, at least about 6 microns, at least about 8 microns, at least about 10 microns, at least about 15 microns, at least about 20 microns, or at least about 25 microns) and/or at most about 100 microns (e.g., at most about 90 microns, at most about 80 microns, at most about 70 microns at most about 60 microns, at most about 50 microns, at most about 40 microns, or at most about 30 microns). In some embodiments, the thickness of the dielectric film is less than about 5 microns (e.g., less than about 4.5 microns, less than about 4.0 microns, less than about 3.5 microns, less than about 3.0 microns, less than about 2.5 microns, or less than about 2.0 microns).

In some embodiments, when the dielectric composition is photosensitive, the process to prepare a patterned photosensitive dielectric film includes converting the photosensitive dielectric film into a patterned dielectric film by a lithographic process. In such cases, the conversion can include exposing the photosensitive dielectric film to high energy radiation (such as electron beams, ultraviolet light, and X-ray) using a patterned mask.

After the exposure, the dielectric film can be heat treated from at least about 50° C. (e.g., at least about 55° C., at least about 60° C., or at least about 65° C.) to at most about 100° C. (e.g., at most about 95° C., or at most about 90° C., at most about 85° C., at most about 80° C., at most about 75° C., or at most about 70° C.) for at least about 60 seconds (e.g., at least about 65 seconds, or at least about 70 seconds) to at most about 240 seconds (e.g., at most about 180 seconds, at most about 120 seconds or at most about 90 seconds). The heat treatment is usually accomplished by use of a hot plate or oven.

After the exposure and heat treatment, the dielectric film can be developed to remove unexposed portions by using a developer to form openings or a relief image on the substrate. Development can be carried out by, for example, an immersion method or a spraying method. Microholes and fine lines can be generated in the dielectric film on the laminated substrate after development.

In some embodiments, the dielectric film can be developed by use of an organic developer. Examples of such developers can include, but are not limited to, the purified bioderived organic solvents described herein, such as gamma-valerolactone, cyrene, 2-methyl tetrahydrofuran and the like. Other solvents include, but are not limited to gamma-butyrolactone (GBL), dimethyl sulfoxide (DMSO), N,N-diethylacetamide, methyl ethyl ketone (MEK), methyl isobutyl ketone (MIBK), 2-heptanone, cyclopentanone (CP), cyclohexanone, n-butyl acetate (nBA), propylene glycol methyl ether acetate (PGMEA), propylene glycol methyl ether (PGME), ethyl lactate (EL), propyl lactate, 3-methyl-3-methoxybutanol, tetralin, isophorone, ethylene glycol monobutyl ether, diethylene glycol monoethyl ether, diethylene glycol monoethyl ether acetate, diethylene glycol dimethyl ether, diethylene glycol methylethyl ether, triethylene glycol monoethyl ether, dipropylene glycol monomethyl ether, methyl 3-methoxypropionate, ethyl 3-ethoxypropionate, diethyl malonate, ethylene glycol, 1,4:3,6-dianhydrosorbitol, isosorbide dimethyl ether, 1,4:3,6-dianhydrosorbitol 2,5-diethyl ether (2,5-diethylisosorbide) and mixtures thereof. Preferred developers are gamma-valerolactone, cyrene, 2-methyl tetrahydrofuran, gamma-butyrolactone (GBL), cyclopentanone (CP), cyclohexanone, ethyl lactate (EL), n-butyl acetate (nBA) and dimethylsulfoxide (DMSO). More preferred developers are gamma-valerolactone, cyrene, 2-methyl tetrahydrofuran, gamma-butyrolactone (GBL), cyclopentanone (CP) and cyclohexanone. These developers can be used individually or in combination of two or more to optimize the image quality for the composition and lithographic process.

In some embodiments, the dielectric film can be developed by using an aqueous developer. When the developer is an aqueous solution, it preferably contains one or more aqueous bases. Examples of suitable bases include, but are not limited to, inorganic alkalis (e.g., potassium hydroxide, sodium hydroxide), primary amines (e.g., ethylamine, n-propylamine), secondary amines (e.g., diethylamine, di-n-propylamine), tertiary amines (e.g., triethylamine), alcoholamines (e.g., triethanolamine), quaternary ammonium hydroxides (e.g., tetramethylammonium hydroxide or tetraethylammonium hydroxide), and mixtures thereof. The concentration of the base employed will vary depending on, e.g., the base solubility of the polymer employed. The most preferred aqueous developers are those containing tetramethylammonium hydroxide (TMAH). Suitable concentrations of TMAH range from about 1% to about 5%.

In some embodiments, after the development by an organic developer, an optional rinse treatment can be carried out with an organic rinse solvent to remove residues. Suitable examples of organic rinse solvents include, but are not limited to, alcohols such as isopropyl alcohol, methyl isobutyl carbinol (MIBC), propylene glycol monomethyl ether (PGME), and amyl alcohol; esters such as n-butyl acetate (nBA), ethyl lactate (EL) and propylene glycol monomethyl ether acetate (PGMEA); ketnoes such as methyl ethyl ketone, and mixtures thereof.

In some embodiments, after the development step or the optional rinse treatment step, an optional baking step (e.g., post development bake) can be carried out at a temperature ranging from at least about 120° C. (e.g., at least about 130° C., at least about 140° C., at least about 150° C., at least about 160° C., at least about 170° C., or at least about 180° C.) to at most about 250° C. (e.g., at most about 240° C., at most about 230° C., at most about 220° C., at most about 210° C., at most about 200° C. or at most about 190° C.). The baking time is at least about 5 minutes (e.g., at least about 10 minutes, at least about 20 minutes, at least about 30 minutes, at least about 40 minutes, at least about 50 minutes, or at least about 60 minutes) and/or at most about 5 hours (e.g., at most about 4 hours, at most about 3 hours, at most about 2 hours, or at most about 1.5 hours). This baking step can remove residual solvent from the remaining dielectric film and can further crosslink the remaining dielectric film. Post development bake can be done in air or preferably, under a blanket of nitrogen and may be carried out by any suitable heating means.

In some embodiments, the patterned dielectric film includes at least one element having a feature size of at most about 10 microns (e.g., at most about 9 microns, at most about 8 microns, at most about 7 microns, at most about 6 microns, at most about 5 microns, at most about 4 microns, at most about 3 microns, at most about 2 microns, or at most about 1 microns). One important aspect of this disclosure is that the dielectric films prepared from the dielectric film-forming composition described herein are capable of producing a patterned film with a feature size of at most about 3 microns (e.g., at most 2 microns or at most 1 micron) by a laser ablation process.

In some embodiments, the aspect ratio (ratio of height to width) of a feature (e.g., the smallest feature) of the patterned dielectric film of this disclosure is at least about 1/3 (e.g., at least about 1/2, at least about 1/1, at least about 2/1, at least about 3/1, at least about 4/1, or at least about 5/1).

In some embodiments (e.g., when the dielectric film-forming composition is non-photosensitive), the process to prepare patterned dielectric film include converting the dielectric film into patterned dielectric film by a laser ablation technique. Direct laser ablation process with an excimer laser beam is generally a dry, one step material removal to form openings (or patterns) in the dielectric film. In some embodiments, the wavelength of the laser is 351 nm or less (e.g., 351 nm, 308 nm, 248 nm or 193 nm). Examples of suitable laser ablation processes include, but are not limited to, the processes described in U.S. Pat. Nos. 7,598,167, 6,667,551, and 6,114,240, the contents of which are hereby incorporated by reference.

In embodiments when the dielectric film-forming composition is non-photosensitive, the composition can be used to form the bottom layer in a bilayer photoresist. In such embodiment, the top layer of the bilayer photoresist can be a photosensitive layer and can be patterned upon exposure to high energy radiation. The pattern in the top layer can be transferred to the bottom dielectric layer (e.g., by etching). The top layer can then be removed (e.g., by using a wet chemical etching method) to form a patterned dielectric film.

In some embodiments, this disclosure features a process for depositing a metal layer (e.g., to create an embedded copper trace structure) that includes the steps of: (a) forming a patterned dielectric film having openings; and d) depositing a metal layer (e.g., an electrically conductive metal layer) in at least one opening in the patterned dielectric film. For example, the process can include the steps of: (a) depositing a dielectric film-forming composition of this disclosure on a substrate (e.g., a semiconductor substrate) to form a dielectric film; (b) exposing the dielectric film to a source of radiation or heat or a combination thereof (e.g., through a mask); (c) patterning the dielectric film to form a patterned dielectric film having openings; and (d) depositing a metal layer (e.g., an electrically conductive metal layer) in at least one opening in the patterned dielectric film. In some embodiments, steps (a)-(d) can be repeated one or more (e.g., two, three, or four) times.

In some embodiments, this disclosure features a process to deposit a metal layer (e.g., an electrically conductive copper layer to create an embedded copper trace structure) on a semiconductor substrate. In some embodiment, to achieve this, a seed layer conformal to the patterned dielectric film is first deposited on the patterned dielectric film (e.g., outside the openings in the film). Seed layer can contain a barrier layer and a metal seeding layer (e.g., a copper seeding layer). In some embodiments, the barrier layer is prepared by using materials capable of preventing diffusion of an electrically conductive metal (e.g., copper) through the dielectric layer. Suitable materials that can be used for the barrier layer include, but are not limited to, tantalum (Ta), titanium (Ti), tantalum nitride (TiN), tungsten nitride (WN), and Ta/TaN. A suitable method of forming the barrier layer is sputtering (e.g., PVD or physical vapor deposition). Sputtering deposition has some advantages as a metal deposition technique because it can be used to deposit many conductive materials, at high deposition rates, with good uniformity and low cost of ownership. Conventional sputtering fill produces relatively poor results for deeper, narrower (high-aspect-ratio) features. The fill factor by sputtering deposition has been improved by collimating the sputtered flux. Typically, this is achieved by inserting between the target and substrate a collimator plate having an array of hexagonal cells.

Next step in the process is metal seeding deposition. A thin metal (e.g., an electrically conductive metal such as copper) seeding layer can be formed on top of the barrier layer in order to improve the deposition of the metal layer (e.g., a copper layer) formed in the succeeding step.

Next step in the process is depositing an electrically conductive metal layer (e.g., a copper layer) on top of the metal seeding layer in the openings of the patterned dielectric film wherein the metal layer is sufficiently thick to fill the openings in the patterned dielectric film. The metal layer to fill the openings in the patterned dielectric film can be deposited by plating (such as electroless or electrolytic plating), sputtering, plasma vapor deposition (PVD), and chemical vapor deposition (CVD). Electrochemical deposition is generally a preferred method to apply copper since it is more economical than other deposition methods and can flawlessly fill copper into the interconnect features. Copper deposition methods generally should meet the stringent requirements of the semiconductor industry. For example, copper deposits should be uniform and capable of flawlessly filling the small interconnect features of the device, for example, with openings of 100 nm or smaller. This technique has been described, e.g., in U.S. Pat. No. 5,891,804 (Havemann et al.), 6,399,486 (Tsai et al.), and 7,303,992 (Paneccasio et al.), the contents of which are hereby incorporated by reference.

In some embodiments, the process of depositing an electrically conductive metal layer further includes removing overburden of the electrically conductive metal or removing the seed layer (e.g., the barrier layer and the metal seeding layer). In some embodiments, the overburden of the electrically conductive metal layer (e.g., a copper layer) is at most about 3 microns (e.g., at most about 2.8 microns, at most about 2.6 microns, at most about 2.4 microns, at most about 2.2 microns, at most about 2.0 microns, or at most about 1.8 microns) and at least about 0.4 micron (e.g., at least about 0.6 micron, at least about 0.8 micron, at least about 1.0 micron, at least about 1.2 micron, at least about 1.4 micron or at least about 1.6 microns). Examples of copper etchants for removing copper overburden include an aqueous solution containing cupric chloride and hydrochloric acid or an aqueous mixture of ferric nitrate and hydrochloric acid. Examples of other suitable copper etchants include, but are not limited to, the copper etchants described in U.S. Pat. Nos. 4,784,785, 3,361,674, 3,816,306, 5,524,780, 5,650,249, 5,431,776, and 5,248,398, and US Application Publication No. 2017175274, the contents of which are hereby incorporated by reference.

Some embodiments describe a process for surrounding a metal structured substrate containing conducting metal (e.g., copper) wire structures forming a network of lines and interconnects with the dielectric film of this disclosure. The process can include the steps of:
a) providing a substrate containing conducting metal wire structures that form a network of lines and interconnects on the substrate.
b) depositing a dielectric film-forming composition of this disclosure on the substrate to form a dielectric film (e.g., that surrounds the conducting metal lines and interconnects; and
c) exposing the dielectric film to a source of radiation or heat or a combination of radiation and heat (with or without a mask).

The above steps can be repeated multiple times (e.g., two, three, or four times) to form a complex multi-layered three-dimensional object.

In some embodiments, this disclosure features a method of preparing a dry film structure. The method can include:
a) coating a carrier substrate (e.g., a substrate including at least one polymeric or plastic film) with a dielectric film-forming composition described herein.
b) drying the coated dielectric film-forming composition to form a dry film; and
c) optionally, applying a protective layer to the dry film.

In some embodiments, the carrier substrate is a single or multiple layer polymeric or plastic film, which can include one or more polymers (e.g., polyethylene terephthalate). In some embodiments, the carrier substrate has excellent optical transparency, and it is substantially transparent to actinic irradiation used to form a relief pattern in the polymer layer. The thickness of the carrier substrate is preferably in the range of at least about 10 μm (e.g., at least about 15 μm, at least about 20 μm, at least about 30 μm, at least about 40 μm, at least about 50 μm or at least about 60 μm) to at most about 150 μm (e.g., at most about 140 μm, at most about 120 μm, at most about 100 μm, at most about 90 μm, at most about 80 μm, or at most about 70 μm).

In some embodiments, the protective layer is a single or multiple layer film, which can include one or more polymers (e.g., polyethylene or polypropylene). Examples of carrier substrates and protective layers have been described in, e.g., U.S. Application Publication No. 2016/0313642, the contents of which are hereby incorporated by reference.

In some embodiments, the dielectric film of the dry film can be delaminated from carrier layer as a self-standing dielectric film. A self-standing dielectric film is a film that can maintain its physical integrity without using any support layer such as a carrier layer. In some embodiments, the self-standing dielectric film is not crosslinked or cured and can include the components of the dielectric film-forming composition described above except for the solvent.

In some embodiments, the dielectric loss tangent or dissipation factor of the film prepared from dielectric film-forming composition of this disclosure measured at 10 GHz, 15 GHZ, and/or 35 GHz is in the range of from at least about 0.001 (e.g., at least about 0.002, at least about 0.003, at least about 0.004, at least about 0.005, at least about 0.01, or at least about 0.05) to at most about 0.1 (e.g., at most about 0.08, at most about 0.06, at most about 0.05, at most about 0.04, at most about 0.02, at most about 0.01, at most about 0.008, at most about 0.006, or at most about 0.005).

In some embodiments, the dielectric film of the dry film structure can be laminated to a substrate (e.g., a semiconductor substrate such as a wafer) using a vacuum laminator at about 50° C. to about 140° C. after pre-laminating of the dielectric film of the dry film structure with a plane compression method or a hot roll compression method. When the hot roll lamination is employed, the dry film structure can be placed into a hot roll laminator, the optional protective layer can be peeled away from the dielectric film/carrier substrate, and the dielectric film can be brought into contact with and laminated to a substrate using rollers with heat and pressure to form an article containing the substrate, the dielectric film, and the carrier substrate. The dielectric film can then be exposed to a source of radiation or heat (e.g., through the carrier substrate) to form a crosslinked photosensitive dielectric film. In some embodiments, the carrier substrate can be removed before exposing the dielectric film to a source of radiation or heat.

Some embodiments of this disclosure describe a process of generating a planarizing dielectric film on a substrate with copper pattern. In some embodiments, the process includes depositing a dielectric film-forming composition onto a substrate with copper pattern to form a dielectric film. In some embodiments, the process includes steps of:
a. providing a dielectric film-forming composition of this disclosure, and
b. depositing the dielectric film-forming composition onto a substrate with copper pattern to form a dielectric film, wherein the difference in the highest and lowest points on a top surface of the dielectric film is less than about 2 microns (e.g., less than 1.5 microns, less than 1 micron or less than 0.5 micron).

In some embodiments, this disclosure features an article containing at least one patterned dielectric film formed by a process described herein. Examples of such articles include a semiconductor substrate, a flexible film for electronics, a wire isolation, a wire coating, a wire enamel, and an inked substrate. In some embodiments, this disclosure features semiconductor devices that include one or more of these articles. Examples of semiconductor devices that can be made from such articles include an integrated circuit, a light emitting diode, a solar cell, and a transistor.

The contents of all publications cited herein (e.g., patents, patent application publications, and articles) are hereby incorporated by reference in their entirety.

The present disclosure is illustrated in more detail with reference to the following examples, which are for illustrative purposes and should not be construed as limiting the scope of the present disclosure.

### Purification Example 1: Purification of GVL

Table 1 below includes the data generated on the gamma-valerolactone (GVL) from Sigma-Aldrich.

TABLE 1

| Parameter | Result | Unit |
|---|---|---|
| Purity (GC) | 99.02 | % |
| Water | 0.07 | % |
| Acidity | 3.30 | μeq/gram |
| Chloride | 0.28 | Ppm |

Trace Metals

| | | |
|---|---|---|
| Al | 1 | Ppb |
| Calcium | 0 | Ppb |
| Chromium | 1 | Ppb |
| Copper | 0 | Ppb |
| Iron | 10 | Ppb |
| Magnesium | 0 | Ppb |
| Manganese | 0 | Ppb |
| Nickel | 1 | Ppb |
| Potassium | 0 | Ppb |
| Sodium | 5 | Ppb |
| Zinc | 12 | Ppb |
| Cobalt | 0 | Ppb |
| Silver | 2 | Ppb |
| Titanium | 0 | Ppb |

### Processing of Purified Bioderived Solvent

During the production of the of Examples and Comparative Examples, the following bioderived organic solvents are prepared: GVL, cymene, and cyrene. For each of the bioderived organic solvents, an organic solvent with a high-purity grade having a purity of 99% by mass or more is used as a raw material for the production of the purified bioderived organic solvent, and in addition, the raw material is purified in advance by distillation, ion exchange, filtering, or the like. Using the raw materials obtained in this manner, each of the bioderived organic solvents is purified using the following steps: (1) an ion exchange treatment step in which an organic solvent is subjected to an ion exchange treatment, (2) a dehydration treatment step in which the organic solvent after the first ion exchange treatment is subjected to dehydration, and (3) a distillation treatment step in which the organic solvent after the dehydration treatment is subjected to distillation.

### General Description of Trace Metal Measurement

The total trace metal concentration in a purified bioderived organic solvent sample is tested by using inductively coupled plasma mass spectrometry (ICP-MS). Using a Fujifilm developed method, each sample is tested for the presence of 36 metal species. The detection limit is metal specific, but the typical detection limits are in the range of 0.00010-100.0 ppb.

### General Description of Trace Moisture and Organic Impurities Measurement

The trace moisture and organic impurities in a purified bioderived solvent sample are measured by using Thermal Desorption-Gas Chromatography/Mass Spectrometry (TD-GC/MS). A small volume of a liquid sample is injected into a thermal desorption tube containing a sorbent and put into a thermal desorber. The sample is heated, then injected into the GC/MS unit where the sample mixture is separated into its components and components are identified by mass.

### General Description of Particle Counts Measurement

The particle counts in a purified bioderived solvent sample is measured by using RION KS 18F.

### Photosensitive Composition Example 1: Preparation of Photosensitive Composition Containing PBO Precursor (PBO-1)

A photosensitive composition was prepared by using 28.48 g of PBO precursor polymer (I):

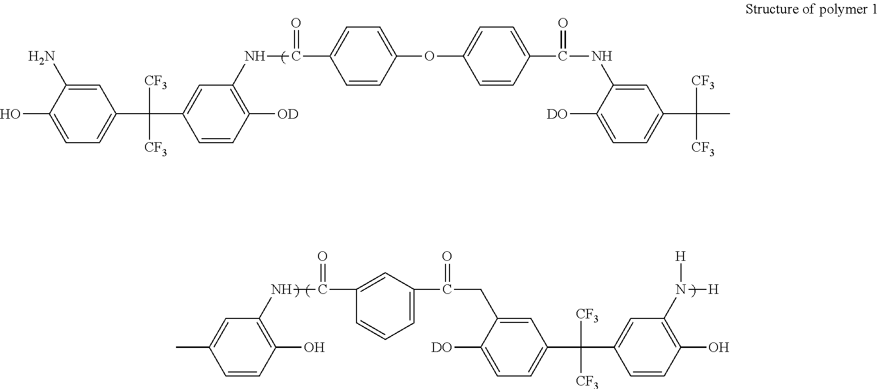

46.10 g of bioderived gamma-valerolactone, 0.87 g of gamma-ureidopropyltrimethoxysilane, 0.70 g of diphenyl silane diol, and 3.85 g of PAC of Structure (II) was prepared. This composition was easily filtered using 0.2 μm filter.

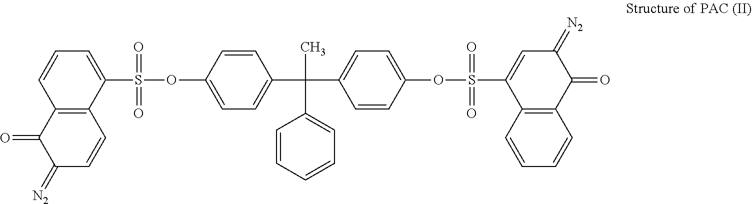

### Comparative Photosensitive Composition Example 1

A photosensitive composition was prepared by using 28.48 g of PBO precursor polymer (I), 46.10 g of gamma-butyrolactone, 0.87 g of gamma-ureidopropyltrimethoxysilane, 0.70 g of diphenyl silane diol, and 3.85 g of PAC of Structure (II). Filtration of this composition using 0.2 μm filter was slow. In order to filter this composition, it was necessary to use first a prefilter before using 0.2 μm filter.

### Synthesis Example 1: Preparation of Polymer FCP-1

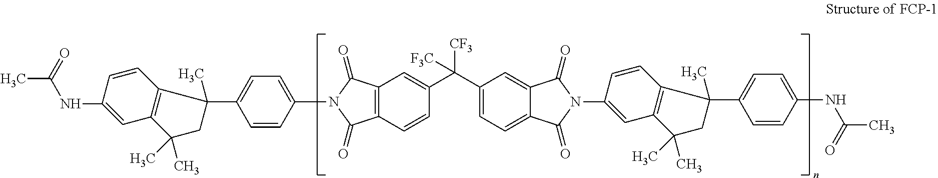

Solid 4,4′-(hexafluoroisopropylidene)bis(phthalic anhydride) (6FDA) (2.370 kg, 5.33 mole) was charged to a solution of 1-(4-aminophenyl)-1,3,3-trimethylindan-5-amine (also known as 4,4′-[1,4-phenylene-bis(1-methylethylidene)]bisaniline (DAPI)) (1.465 kg, 5.51 mole) in NMP (9.86 kg) at 25° C. The reaction mixture temperature was increased to 40° C. and allowed to react for 6 hours. Next, acetic anhydride (1.125 kg) and pyridine (0.219 kg) were added and the reaction mixture temperature was increased to 100° C. and allowed to react for 12 hours.

The reaction mixture above was cooled to room temperature and transferred to a larger vessel equipped with a mechanical stirrer. The reaction solution was diluted with ethyl acetate and washed with water for one hour. After the stirring was stopped, the mixture was allowed to stand undisturbed. Once phase separation had occurred, the aqueous phase was removed. The organic phase was diluted with a combination of ethyl acetate and acetone and washed twice with water. The amounts of organic solvents (ethyl acetate and acetone) and water used in all of the washes are shown in Table 2.

TABLE 2

| | Wash 1 | Wash 2 | Wash 3 |
|---|---|---|---|
| Ethyl Acetate (kg) | 20.5 | 4.1 | 4.1 |
| Acetone (kg) | — | 2.3 | 2.3 |
| Water (kg) | 22.0 | 26.0 | 26.0 |

Cyclopentanone (10 kg) was added to the washed organic phase and the solution was concentrated by vacuum distillation to give a polymer solution FCP-1. The solid % of final polymer was 29.19% and the weight average molecular weight (Mw) measured by GPC was 54,000 Daltons.

### Synthesis Example 2: Preparation of Fully Cyclized Polyimide (1)

The following is an example of preparation of a polyimide (PI) polymer using one diamine and one dianhydride wherein the isolation solvent (i.e., a lactone) was different from the purification solvents (i.e., a ketone and an ester).

Solid 4,4′-oxidiphthalic anhydride (ODPA, 664.5 g) was charged to a solution of 4,4′-diamino-2,2′-bis(trifluoromethyl) biphenyl (TFMB, 722.1 g) in NMP (3296 g) at 25° C. Additional NMP (1346 g) was used to rinse the dianhydride into solution. The reaction temperature was increased to 40° C. and the mixture was allowed to react for 3 hours. Next, acetic anhydride (507.2 g) and pyridine (98.3 g) were added, the reaction temperature was increased to 100° C., and the mixture was allowed to react for 12 hours.

The reaction mixture was cooled to room temperature and a portion (899 g) was transferred to a 5-L vessel equipped with a mechanical stirrer. The reaction solution was diluted using a combination of cyclopentanone and n-butyl acetate and washed with water for one hour. Stirring was stopped and the mixture was allowed to stand undisturbed. Once phase separation had occurred, the aqueous phase was removed. The organic phase was diluted using cyclopentanone and washed three more times with water. The amounts of purification solvents (i.e., cyclopentanone and n-butyl acetate) and water used in all of the washes are shown in Table 3.

TABLE 3

| | Wash 1 | Wash 2 | Wash 3 | Wash 4 |
|---|---|---|---|---|
| Cyclopentanone (g) | 1385 | 202 | — | — |
| n-Butyl Acetate (g) | 892 | — | — | — |
| Water (g) | 1329 | 1628 | 1631 | 1630 |

The washed organic phase was concentrated by vacuum distillation. Gamma-valerolactone (605 g) was added as an isolation solvent and vacuum distillation was continued. The final polymer solution had a concentration of 24.99 wt %.

### Synthesis Example 2: Preparation of Fully Cyclized Polyimide (2)

Solid ODPA (14.73 g) was charged to a solution of TFMB (16.01 g) in 1:1 bioderived gama-valerolactone:cyrene (73.18 g) at 25° C. Additional 1:1 bioderived gama-valerolactone:cyrene (29.75 g) was used to rinse the dianhydride into solution. The reaction temperature was increased to 40° C. and the mixture was allowed to react for 3 hours. Next, acetic anhydride (11.32 g) and pyridine (2.21 g) were added, the reaction temperature was increased to 100° C., and the mixture was allowed to react for 12 hours.

### Synthesis Example 3: Preparation of Fully Cyclized Polyimide (3)

A mixture of solid ODPA (94.78 g) and 2,2-[bis(3, 4-dicarboxyphenyl)] hexafluoropropane dianhydride (6FDA) (45.25) was charged to a solution of TFMB (135.5 g) in NMP (819 g) at 25° C. Additional NMP (100 g) was used to rinse the dianhydride into solution. The reaction temperature was increased to 40° C. and the mixture was allowed to react for 3 hours. Next, acetic anhydride (94.25 g) and pyridine (18.27 g) were added, the reaction temperature was increased to 100° C., and the mixture was allowed to react for 12 hours.

### Photosensitive Composition Example 2: Preparation of Photosensitive Composition PSC-1

A photosensitive dielectric film-forming composition was prepared by using 36.65 parts of a polyamic acid ester produced from 4,4′-oxidiphthalic anhydride (ODPA), 4,4′-diaminophenyl ether (ODA) and 2-hydroxyethyl methacrylate (Durimide 733), 5.5 parts of 3,6,9-trioxaundecamethylene dimethacrylate, 0.73 parts of 3-(triethoxysilyl) propylsuccinic anhydride, 0.88 parts of 1-[4-(phenylthio)phenyl]-1,2-octanedione 2-(O-benzoyloxime) (OXE01 from BASF), 0.073 parts of monomethyl ether hydroquinone, 0.060 tetrazole (5% solution in GBL/DMSO); 22.2 parts of dimethyl sulfoxide, 88.8 parts of bioderived gamma-veralolactone (GVL) commercially available from Sigma-Aldrich. After being stirred mechanically for 24 hours, the solution was filtered by using a 0.2-micron filter (Ultradyne from Meissner Corporation, cat #CLTM0.2-552).

### Photosensitive Composition Example 3: Preparation of Photosensitive Composition PSC-2

A photosensitive dielectric film-forming composition (PSC-2) was prepared by using 100 parts of a 29.19% a solution of a polyimide polymer (FCP-1) having a weight average molecular weight of 54,000 Daltons in cyclopentanone, 2.76 parts of cyclopentanone, 41.5 parts of bioderived gamma-valerolactone (GVL) commercially available from Sigma-Aldrich as listed in Purification Example 1, 1.75 parts of a 0.5 wt % solution of PolyFox 6320 (a surfactant available from OMNOVA Solutions) in cyclopentanone, 1.46 parts of methacryloxypropyltrimethoxy silane (an adhesion promoter), 0.88 parts of 2-(O-benzoyloxime)-1-[4-(phenylthio)phenyl]-1,2-octanedione (Irgacure OXE-1 available from BASF, a photoinitiator), 0.06 parts of monomethyl ether hydroquinone (an antioxidant), 10.95 parts of tetraethylene glycol diacrylate (a reactive functional compound), 3.65 parts of pentaerythritol triacrylate (a reactive functional compound), 2.92 parts of 2,2-bis(4-cyanatophenyl) propane (a cyanate ester) and 0.15 parts of 5-methyl benzotriazole (a copper corrosion inhibitor). After being stirred mechanically for 24 hours, the solution was filtered by using a 0.2-micron filter (Ultradyne from Meissner Corporation, cat #CLTM0.2-552).

The solution is submitted for trace metal analysis. All trace metals are below 500 ppb, which meets the requirement of employing such solution for semiconductor packaging application.

### Dry Film Example 1

A photosensitive dielectric film-forming composition is prepared by using 1345.24 g of a 31.69% solution of a polyimide polymer (FCP-1) having a weight average molecular weight of 57000 in cyclopentanone, 1021.91 g of bioderived gamma valerolactone (GVL) as prepared by Purification Example 1, 102.31 g of a 0.5 wt % solution of PolyFox 6320 in cyclopentanone, 21.31 g of methacryloxypropyltrimethoxy silane, 34.11 g 50% solution of XU-378 (Bisphenol M Cyanate ester available from Huntsman) in cyclopentanone, 12.79 g of Irgacure OXE-1, 0.43 g of monomethyl ether hydroquinone, 138.55 g of tetraethylene glycol diacrylate, 53.39 g of pentaerythritol triacrylate, 21.32 g of ethylene glycol dicyclopentenyl ether acrylate, 4.26 g of dicumyl peroxide and 0.426 g of 5-methyl benzotriazole. After being stirred mechanically for 24 hours, the solution is filtered by using a 0.2-micron filter (Ultradyne from Meissner Corporation, cat #CLTM0.2-552).

This photosensitive dielectric film-forming composition is applied using a slot die coater with a line speed of about 2 feet/minutes (61 cm per minutes) with 60 microns clearance onto a polyethylene terephthalate (PET) film (TCH21, manufactured by DuPont Teijin Films USA) having a width of 16.2″ and thickness of 36 microns used as a carrier substrate and dried at 194° F. to obtain a photosensitive polymeric layer with a thickness of approximately 12.0 microns. On this polymeric layer, a biaxially oriented polypropylene film having width of 16″ and thickness of 30 microns (BOPP, manufactured by Impex Global, Houston, TX) is laid over by a roll compression to act as a protective layer. The carrier substrate, the photosensitive polymeric layer, and the protective layer together formed a dry film (i.e., DF-1)

### Example: Formation of Three-Dimensional Object

The photosensitive dielectric film-forming composition prepared in Dry Film Example 1 is converted to films deposited on various substrates used in microelectronics and packaging applications. A film is deposited on a 100 mm silicon wafer by spin coating about 5 g of the solution at spin speed of about 2000 rpm. The film is dried at a temperature of 105° C. for 3 minutes on a hotplate. A 12-micron clear and transparent film is obtained. Film thickness uniformity across 100 mm substrate is within 0.5 micron. Total particle defect count of the film is below 500 particles/cm2. Film quality in terms of transparency, defect count, and uniformity meets the requirement of employing such a solution for semiconductor packaging application.

The above tests are repeated on aluminum, copper, and silicon nitride. All films thus obtained meet the requirement of employing such a solution for semiconductor packaging application.

Other embodiments are in the claims.

What is claimed is:

1. A method of purifying a bioderived organic solvent, comprising:
(1) passing the bioderived organic solvent through an ion exchange filter unit, wherein the ion exchange filter unit includes a housing and at least one first ion exchange filter and at least one second ion exchange filter in the housing, wherein the at least one first ion exchange filter and the at least one second ion exchange filter are both negatively charged ion exchange filters and are connected in series, and wherein the at least one first ion exchange filter is different from the at least one second ion exchange filter;
(2) passing the bioderived organic solvent through at least one column containing an adsorbent to remove water in the bioderived organic solvent; and
(3) distilling the bioderived organic solvent in a distillation column to obtain a purified bioderived organic solvent;
wherein the bioderived organic solvent is obtained from a biological feedstock, the purified bioderived organic solvent comprises an acid component in an amount of from about 0.1 ppb by mass to about 1000 ppm by mass, and the purified bioderived organic solvent comprises water in an amount of from about 0.1 ppb by mass to about 1000 ppm by mass.

2. The method of claim 1, wherein the bioderived organic solvent comprises a lactone, a ketone, an ether, an alkyl aromatic, an alcohol, or an alkyl alicyclic.

3. The method of claim 2, wherein the bioderived organic solvent comprises gamma-valerolactone, cyrene, 2-methyl tetrahydrofuran, glycerol, pinene, limonene, or cymene.

4. The method of claim 2, wherein the lactone is gamma-valerolactone.

5. The method of claim 2, wherein the ketone is cyrene.

6. The method of claim 2, wherein the ether is 2-methyl tetrahydrofuran.

7. The method of claim 1, wherein the biological feedstock is selected from the group consisting of lignocellulosic biomasses, sugar cane, corn, vegetable oils, oil wastes, or citrus wastes.

8. The method of claim 1, wherein the purified bioderived organic solvent comprises a metal component in an amount of from about 10 ppt by mass to about 500 ppb by mass.

9. The method of claim 1, wherein the purified bioderived organic solvent comprises a total amount of metal components from about 50 ppt to about 500 ppb.

10. The method of claim 1, wherein the bioderived organic solvent is a lactone or an ether, and the purified bioderived organic solvent comprises an acid component in an amount of from about 0.1 ppb by mass to about 100 ppb by mass.

11. The method of claim 1, wherein the bioderived organic solvent comprises water in an amount of from about 10 ppb by mass to about 100 ppm by mass.

12. A purified bioderived organic solvent prepared from the method of claim 1.

13. A purified bioderived organic solvent, wherein
the bioderived organic solvent is obtained from a biological feedstock;
the bioderived organic solvent comprises an acid component in an amount of from about 0.1 ppb by mass to about 1000 ppm by mass; and
the bioderived organic solvent comprises water in an amount of from about 0.1 ppb by mass to about 1000 ppm by mass.

14. The organic solvent of claim 13, wherein the bioderived organic solvent comprises a lactone, a ketone, an ether, an alkyl aromatic, an alcohol, or an alkyl alicyclic.

15. The organic solvent of claim 14, wherein the bioderived organic solvent comprises gamma-valerolactone, cyrene, 2-methyl tetrahydrofuran, glycerol, pinene, limonene, or cymene.

16. A method of preparing a polymer, comprising:
forming a polymer comprising a polyimide precursor polymer, a polybenzoxazole precursor polymer, or a fully imidized polyimide polymer in a solvent system containing at least one first organic solvent and optionally, at least one second organic solvent;
wherein the at least one first organic solvent comprises the purified bioderived organic solvent of claim 13, the bioderived organic solvent is an aprotic polar solvent selected from the group consisting of lactones, ketones, ethers, alkyl aromatics, and alkyl alicyclics, and the at least one second organic solvent comprises a carbonyl group.

17. A photosensitive composition, comprising
at least one resin selected from the group consisting of epoxy resins, novolak resins, polyamide resins, polybenzoxazole precursor polymers, polyimide precursor polymers, and fully imidized polyimide polymers; and
a solvent system containing at least one first organic solvent and optionally, at least one second organic solvent;
wherein the at least one first organic solvent comprises the purified bioderived organic solvent of claim 13, the bioderived organic solvent is a solvent selected from the group consisting of lactones, ketones, ethers, alcohols, alkyl aromatics, and alkyl alicyclics, and the at least one second organic solvent comprises a carbonyl group.

18. The composition of claim 17, further comprising at least one cyanate ester compound, the at least one cyanate ester compound comprising at least two cyanate groups.

19. The composition of claim 17, further comprising at least one reactive functional compound.

20. The composition of claim 19, wherein the at least one reactive functional compound is a di(meth)acrylate containing cross-linker in an amount of from about 5 percent to about 50 percent by weight of the resin.

21. A pattern forming method, comprising:
(a) disposing a photosensitive composition on a substrate to form a film;
(b) exposing the film to radiation, heat, or a combination thereof; and
(c) developing the exposed film using an organic developer to form a pattern on the substrate;
wherein the organic developer comprises the purified bioderived organic solvent of claim 13.
